(19) United States
(12) Patent Application Publication
(10) Pub. No.: US 2021/0251581 A1
(43) Pub. Date: Aug. 19, 2021

(54) Standardization Of Positron Emission Tomography Based Images

(71) Applicant: The Trustees of the University ofPennsylvania, Philadelphia, PA (US)

(72) Inventors: Jayaram K. Udupa, Philadelphia, PA (US); Aliasghar Mortazi, Philadelphia, PA (US); Yubing Tong, Springfield, PA (US); Drew A. Torigian, Philadelphia, PA (US); Dewey Odhner, Horsham, PA (US)

(21) Appl. No.: 17/175,655

(22) Filed: Feb. 13, 2021

Related U.S. Application Data
(60) Provisional application No. 62/976,570, filed on Feb. 14, 2020.

(51) Int. Cl. A61B 6/03; A61B 6/00

(52) U.S. Cl. CPC A61B 6/037; A61B 6/582

(57) ABSTRACT

Methods and systems are described for processing images. An example method may comprise receiving a plurality of images based on positron emission tomography, determining, based on the plurality of images, a plurality of calibration parameters indicative of standardized intensity values for corresponding percentiles of intensity values, determining at least one image associated with a patient. The method may comprise applying, based on the plurality of calibration parameters, a transformation to the at least one image associated with the patient. The method may comprise providing the transformed at least one image. A model may be determined based on a plurality of transformed images. The model may be used to determine an estimated disease burden of an anatomic region.

### CROSS-REFERENCE TO RELATED APPLICATIONS

This application claims priority to and is a non-provisional of U.S. Patent Application No. 62/976,570 filed Feb. 14, 2020, which is hereby incorporated by reference for any and all purposes.

### BACKGROUND

Tissue radiotracer activity measured from positron emission tomography (PET) images is an important biomarker that is clinically utilized for diagnostic, staging, prognostication, and treatment response assessment purposes in patients with cancer and other non-neoplastic disorders. There have been several attempts to solve the non-standardization in PET images due to variations in patient-related and technical factors. The widely utilized standardization method of standardized uptake value (SUV) has only compensated for some of the undesired variability related to such parameters, leading to errors in quantification from PET images. Thus, there is a long-felt need for a new standardization method for PET images to further minimize errors in PET quantification.

### SUMMARY

Methods and systems are described for processing images. An example method can comprise receiving a plurality of images based on positron emission tomography, determining, based on the plurality of images, a plurality of calibration parameters indicative of standardized intensity values for corresponding percentiles of intensity values, determining at least one image associated with a patient. The method can comprise applying, based on the plurality of calibration parameters, a transformation to the at least one image associated with the patient. The method can comprise providing the transformed at least one image.

Another example method can comprise determining an image of a subject, wherein the image has intensity values that are standardized based on one or more calibration parameters. The method can comprise determining an indication of an anatomic region in the image. The method can comprise determining, based on the indication of the anatomic region and a model associated with the anatomic region, an indication of a disease burden associated with the image. The method can comprise causing output of the indication of the disease burden associated with the image.

Another example method can comprise determining a plurality of images associated with a plurality of subjects and calibrated based on one or more calibration parameters. The method can comprise determining, based on the plurality of images, a model associated with the anatomic region. The method can comprise causing output of one or more of the model or data based on the model.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

Additional advantages will be set forth in part in the description which follows or may be learned by practice. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1 shows the process of mapping a given image to standard scale.

FIG. 2 shows CV curves among healthy scan data set for liver in sPET, PET, sSUV, SUV, PET_{G}, PET_{z}, SUV_{G}, and S_{UV} images.

FIG. 3 shows CV curves among healthy scan data set for spleen in sPET, PET, sSUV, SUV, PET_{G}, PET_{z}, SUV_{G}, and S_{UV} images.

FIG. 4 shows SD curves among livers in repeated scan data set for sPET, PET, sSUV, SUV, PET_{G}, PET_{z}, SUV_{G}, and S_{UV} images.

FIG. 5 shows SD curves among spleens in repeated scan data set for sPET, PET, sSUV, SUV, PET_{G}, PET_{z}, SUV_{G}, and S_{UV} images.

FIG. 6A shows a histogram of healthy livers of some randomly chosen subjects from PET.

FIG. 6B shows a histogram of a scaled PET by linearly mapping of maximum intensity to 6000.

FIG. 6C shows a histogram of a scaled PET by linearly mapping %96.5 percentile to a maximum fixed value of 5000.

FIG. 6D shows a histogram of an sPET.

FIG. 7A shows a histogram of healthy livers of some randomly chosen subjects from an SUV.

FIG. 7B shows a histogram of a scaled SUV by linearly mapping of maximum intensity to 6000.

FIG. 7C shows a histogram of a scaled SUV by linearly mapping %95.6 percentile to a maximum fixed value of 5000.

FIG. 7D shows a histogram of a scaled sSUV.

FIG. 8 is a block diagram illustrating an example computing device.

FIG. 9 shows an example SUV modeling process.

FIG. 10 shows a cumulative histogram of the standardized SUVs within the esophagus obtained from PET/CT scans of 33 normal subjects and the fit Gaussian function.

FIG. 11 shows an example disease quantification process.

FIG. 12A shows total lesion burden for normal subjects for the right lung.

FIG. 12B shows total lesion burden for normal subjects for the left lung.

FIG. 12C shows total lesion burden for normal subjects for the esophagus.

### DETAILED DESCRIPTION OF ILLUSTRATIVE EMBODIMENTS

Disclosed herein are methods and systems for standardization of PET (sPET). We also propose a new method to select the standardization landmarks for PET images in order to minimize coefficient of variation among the metabolic activities of a set of healthy organs. The sPET method is independent from patient-related and technical factors, making it practical for use in the clinical and research settings.

It was observed that the disclosed sPET method described herein decreases the variation of metabolic activity measurements among a set of healthy organs more than the SUV method does. The sPET method also decreases differences in the metabolic activity of organs measured from repeat PET scans more than the SUV method does. Furthermore, application of our standardization method to both SUV images (sSUV) and PET images (sPET) yields very similar results.

The disclosure describes a new method of standardizing PET images, called sPET, which decreases the variability in PET images without requiring information about patient-related or technical factors, and which works as well as or better than SUV and sSUV methods that do require such information. As such, this method can be useful for improving PET quantification in clinical and research practice.

### 1. Introduction

1.1 Background and Rationale

Cancer is the second cause of death in the United States and is a significant health problem worldwide. In 2019, about 1.8 million new cancer cases and about 0.6 million cancer deaths were reported in the United States [3]. Positron emission tomography (PET), a non-invasive molecular imaging tool, is one of the major clinical imaging techniques used routinely for comprehensive body-wide diagnostic assessment of patients with cancer and other non-cancerous disorders. PET detects, measures, and localizes gamma rays emitted from annihilation events between positrons (emitted by administered positron-emitting isotopes) and electrons, providing for a method to distinguish tissues that have differential radiotracer activities. For example, abnormal changes in tissue metabolic activity can be detected with ¹⁸F-fluorodeoxyglucose (FDG)-PET imaging before structural changes are detectable with computed tomography (CT) or magnetic resonance imaging (MRI). As such, metabolic activity measured from FDG-PET is an important biomarker that is clinically utilized for diagnostic, staging, prognostication, and treatment response assessment purposes in patients with cancer [4, 5, 6, 7].

Although qualitative assessment of PET images in clinical practice is routinely performed, quantitative assessment is encouraged to decrease inter-reader variability and to improve diagnostic performance of study interpretation. In one of the attempts for disease quantitative assessment in PET images, the percent of administered dose per gram of tissue was used as a measure of tumor uptake [8]. However, after comparing this metric between different patients, it was discovered that this value is affected by the patient size as well as by the radiotracer dose administered. To compensate for these factors, another quantitative measurement was introduced [9] called standardized uptake value (SUV), which is the decay-corrected tissue activity concentration of radiotracer in a region of interest (ROI) divided by the injected radiotracer dose per unit body weight (or alternatively body surface area or lean body mass) (see Equation 1). SUV measurement has been widely utilized for semi-quantitative PET assessment in clinical practice given its ease of use.

TABLE 1
Undesired factors affecting metabolic activity in healthy organs
Factors Affecting Metabolic Activity

| Subject-related | Technical |
|---|---|
| Body weight | Duration of radiotracer uptake period |
| Body composition | Partial-volume effects |
| Body habitus | Size of the region of interest (ROI) |
| Serum glucose | Non-uniformity of radiotracer distribution in the |
| levels | tissue or lesion |
| | Attenuation correction and reconstruction methods |
| | Non-optimized image acquisition |
| | Imaging system parameters |

The formula used to calculate SUV (based on body weight) is given in equation 1:

$$SUV = \frac{tissue activity concentration}{injected radiotracer dose / body weight} \qquad ( 1 )$$

where tissue activity concentration (i.e., tissue activity per volume) is the same as PET value, and activity A(t) at time t is calculated from equation 2:

$$A ( t ) = A ( 0 ) * {e}^{- ln 2 * ( \frac{t - {t}_{0}}{{t}_{2}^{1}} )} \qquad ( 2 )$$

where t₀ is the initial start time, A(0) is the activity at the initial start time, and t_{1/2} is radionuclide half-life.

Although SUV partially compensates for certain factors such as patient body weight and administered radiotracer dose, there are many other uncompensated factors that can adversely affect the accurate and precise measurement of tissue radiotracer uptake in PET images. In general, such factors can be divided into two categories: Patient-related factors and technical factors. Patient-related factors include differences in body weight, body composition, body habitus, serum glucose levels, etc. Technical factors include differences in radiotracer uptake period, partial volume effects, ROI size and placement on PET images, image acquisition methods, attenuation correction and image reconstruction methods, etc. [10, 11, 12, 5]. Such uncompensated factors can make accurate and reproducible disease quantification in PET images very challenging, potentially leading to diagnostic errors during disease staging and response assessment that can adversely affect patient management and outcome.

1.2 Related Works

Having metabolic activity of normal organs as control cases can be very useful in disease quantification in PET imaging. As it is discussed by Engel et al. [22], disease quantification and diagnosis should be easier and accurate with having the knowledge about metabolic activity values from healthy organs. Several methods have been proposed for PET or SUV standardization. Some of these methods are performed at the scan acquisition level (e.g., by using a phantom, by modifying image reconstruction, by standardizing the parameters of scan image acquisition (slice thickness, table position time, etc.)). For some other methods, the standardization is done in patient level by controlling and correcting blood glucose levels, restricting the amount of radiotracer dose administered, restricting the allowable delay time for radiotracer uptake, etc. Lastly, for some additional methods, standardization is performed at the image post-processing level by using various methods such as digital PET phantoms.

For example, standardization of the PET/CT scanners was done by phantom tests in patients with peripheral T-cell lymphoma [23]. In another attempt to do standardization by use of a phantom, a simple cylindrical phantom based on resolution and noise measurements was used [24]. DiFilippo et al. developed software to register PET images to a phantom template and to compute ROI measurements of hot vial activity (SUVmax) and background activity (SUVmean) automatically [25]. Fretteli et al. investigated an alternative methodology by using a single reconstruction data set together with a post-reconstruction algorithm for SUV harmonization to solve the problems of increasing SUV values in point-spread function (PSF) corrections in PET/CT reconstruction [18].

Although using scan acquisition level and patient level standardization can help to remove many of the undesired factors from PET images, for each set of new patients, the details of the methods used in each scenario needs to be provided, which is a tedious task and clinically not practical. In contrast, the image post-reconstruction standardization methods are more generic as they can be applied to different kinds of images. For example, an anatomical standardization method is applied to PET images after building a normal model from SUV for determination of the SUV scores as Z-scores for measuring the abnormality of an FDG-PET scan image [26]. In other work, the optimal transformation for producing normal distributions of tumor SUVs was identified by iterating the BoxCox transformation parameter and selecting the parameter that maximized the ShapiroWilk P-value. Optimal transformations were identified for tumor SUVmax distributions at both pre- and post-treatment time points [27]. Orlhac et al. investigated the effect of a genomic harmonization method (ComBat) to normalize radiomic features as measured in PET for removing the center effect while retaining the pathophysiologic information [28]. In another work, a digital reference object (DRO), which is a phantom-like object developed by the Quantitative Imaging Biomarker Alliance (QIBA) FDG-PET technical committee, was used for the purpose of harmonizing SUVs in Tc-99m single photon emission computed tomography/computed tomography (SPECT/CT) imaging [29].

As standardization can potentially improve image interpretation tasks such as disease detection and diagnosis, it has been used in other modalities such as magnetic resonance imaging (MRI). One of the successful standardization methods and the first of its kind was proposed by Nyul et al. [1, 2] for MR image standardization by finding some landmarks at different percentiles (such as median and mean) from a histogram and then making a model from the obtained landmarks from calibration images. Then, all intensity values of a given image will be mapped non-linearly by using the landmarks. In another attempt, Madabhushi et al. [30] proposed a standardization method which can handle non-similarity among intensity values in the same tissues in different subject due to differences in histogram-based landmarks. Furthermore, Zhuge et al. [31] proposed an intensity-based standardization for correcting background inhomogeneity in MR images.

In this disclosure, a new standardization method for PET images, called sPET, is proposed. As previously discussed, one of the major challenges for finding the landmarks is finding a proper restriction for the tail of the histogram. The histogram tail (which represents the high intensity values in images) is usually due to artifacts and noise in the image. Nyul et al. [1] proposed to consider standard deviation and its changes (derivatives) among the maximum percentile of training/calibration images in histograms to find the proper maximum percentile [1, 2]. In the current study, we introduce a new setting to remove the high intensity values from histograms. Also, we propose new evaluation metrics to compare and evaluate different standardization methods in PET images. The description is organized as follows: In section 2, the overview of proposed method and its details and the evaluation methods are explained. The experiments and data sets which are used are explained in section 3. Then, the discussion and conclusion are mentioned in section 4.

### 2. Materials and Methods

2.1 Overview and Notations

Let I={I₁, I₂, . . . , I_{N}} be a set of 3D PET images (stack of all PET image slices) of a body region B. For any given image I in I let I_{P} be a corresponding PET image (based on activity concentration values) and let I_{S} be a corresponding SUV image (based on the SUV values as defined in Equation 1). sI is used to indicate the standardized I in I and accordingly sI_{S} or sI_{P} are used to indicate a standardized SUV (sSUV) image and a standardized PET (sPET) image, respectively. For any image I in I, let its minimum and maximum intensities be denoted by min(I) and max(I), and let pₘᵢₙ(I), med(I), and pₘₐₓ(I) denote the minimum (e.g., low) percentile, median (50^{th}) percentile, and maximum (e.g., high) percentile intensities of I, respectively. The proposed standardization method is based on first defining a standardization scale which involves finding landmarks pₘᵢₙ(I), med(I), and pₘₐₓ(I) on the histograms of a subset I_{c} of I used for calibration and finding their mean position over the landmarks in the images in I_{c}. Our intent is that the images in I_{c} should be normal (the healthiness of the target organs are confirmed with an expert radiologist in this data set). Subsequently, for any given image I in another subset I_{T} of I to be standardized, which is disjoint from I_{c} and may correspond to normal subjects or subjects with disease, the same landmarks are determined in I, the mapping that results when the landmarks of I are matched to the mean landmarks from the calibration step is computed, and I's voxel intensities are transformed according to the mapping. The proposed method of standardization is explained in greater detail in the following subsections in terms of: 1) finding the landmarks from histograms, 2) applying the standardization scale to the given images, and 3) evaluating and comparing amongst the different standardization methods.

2.2 Finding Landmarks

First, the three landmarks are estimated in all images in I_{c}. Note that the median intensity med(I) is estimated for the foreground (body region) only after removing background voxels as described in [1]. Then, for the three landmark intensities, their mean values, denoted respectively by sₘᵢₙ, sₘ, sₘₐₓ, over the images in I_{c} are determined as following:

$${s}_{min} = \frac{1}{C} \mathring{{a}_{{IÎI}_{c}}} {p}_{min} ( I ) , \\ {s}_{m} = \frac{1}{C} \mathring{{a}_{{IÎI}_{c}}} med ( I ) , \\ {s}_{max} = \frac{1}{C} \mathring{{a}_{{IÎI}_{c}}} {p}_{max} ( I ) , \qquad ( 3 )$$

where C is the number of images in I_{c}. These values define the standardized scale. In our application, typically pₘᵢₙ(I)=min(I) (after removing the background or after removing the first peak in the histogram). However, we chose pₘₐₓ(I)≠max(I), since this inequality plays a vital role in standardization. The reason for this choice is that the tail (or maximum) of the histogram of I is affected by artifacts and outlier intensities which cause significant variation among subjects and scanners. As we show such variations in PET can lead to undesired SUV variations among healthy organs from different subjects and scanners. Following [2, 1] to solve this problem, we use pₘᵢₙ(I) and pₘₐₓ(I) as landmarks such that only within the interval [pₘᵢₙ(I), pₘₐₓ(I)] we seek to uniformize intensity meaning across subjects. This implies that normal tissues and organs are least affected by non-standardness after standardization, although outlier intensities in the histogram tail are transformed faithfully.

One of the main challenges and a novelty in this standardization method is to find the appropriate maximum percentile pₘₐₓ(I) for removing the adverse effect from the tail of the histogram in the standardization procedure. In order to find the appropriate pₘₐₓ(I), deviating from [2], we utilize a different method as follows. We define the mean metabolic activity MMA(O) derived from the SUV image I_{S} of an object O as:

$$MMA ( O ) = \frac{TMA ( O )}{{\sum }_{v} \vert v \vert \times {FM}_{0} ( v )} , \qquad ( 4 )$$

where FM_{O} is the binary mask of O, FM_{O}(v) is the binary mask value (0 or 1) at voxel denotes the volume of voxel v, and TMA(O) is the total metabolic activity of O defined as:

$$TMA ( O ) = {\sum }_{v} \vert v \vert \times {FM}_{O} ( v ) \times {I}_{S} ( v ) , \qquad ( 5 )$$

Our argument is that any healthy organ (or tissue region) O should have a very similar MMA(O) among different subjects. Thus, we expect that after using any standardization method, the coefficient of variation (defined in subsection 2.4) of the computed MMA(O) (CV_{MMA}) across different subjects should be as small as possible. CV_{MMA} can then be used as a metric to investigate how to choose the optimum value of pₘₐₓ for the standardization method, and can also be used as a metric to assess which standardization method is better. Along similar lines, instead of using the SUV (or sSUV) image I_{S}, we define the mean activity MA(O) derived from the PET (or sPET) image I_{P} by simply taking the mean image intensity value of I_{P} within the mask of O and the corresponding coefficient of variation CV_{MA} of MA(O) over different samples of O from different subjects.

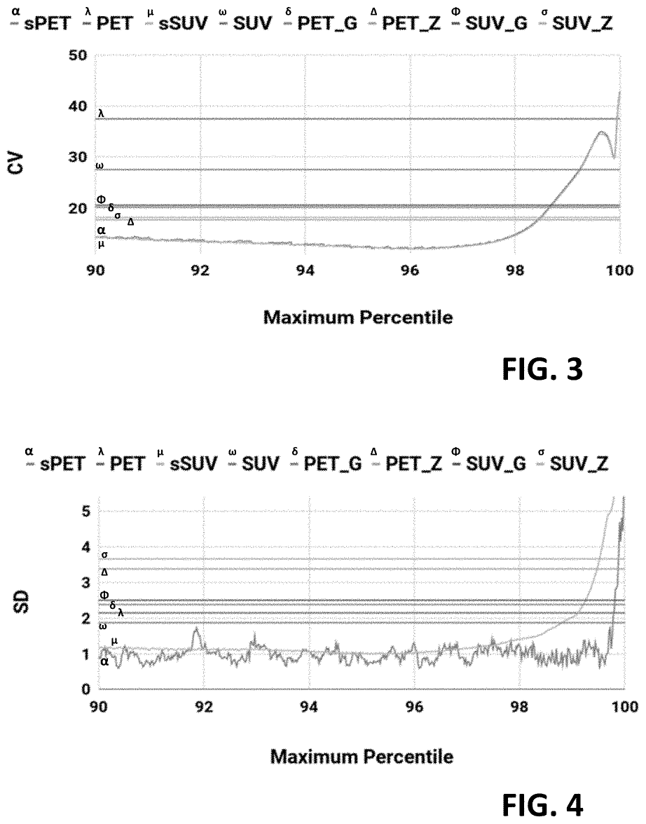

FIGS. 2 and 3 illustrate the variation of CV_{MMA} and CV_{MA} as a function of pₘₐₓ for performing calibration for standardization based separately on I_{S} and IP, respectively, for O=liver and O=spleen over data set I_{c}. The idea is to select pₘₐₓ where CV_{MMA} or CV_{MA} is minimal.

In our approach, we use the healthy scan data set I_{c} to perform all calibration operations and to estimate the parameters of the standardization mapping. Subsequently, the derived mapping can be applied to any other data sets such as I_{T}.

2.3 Applying Standardization

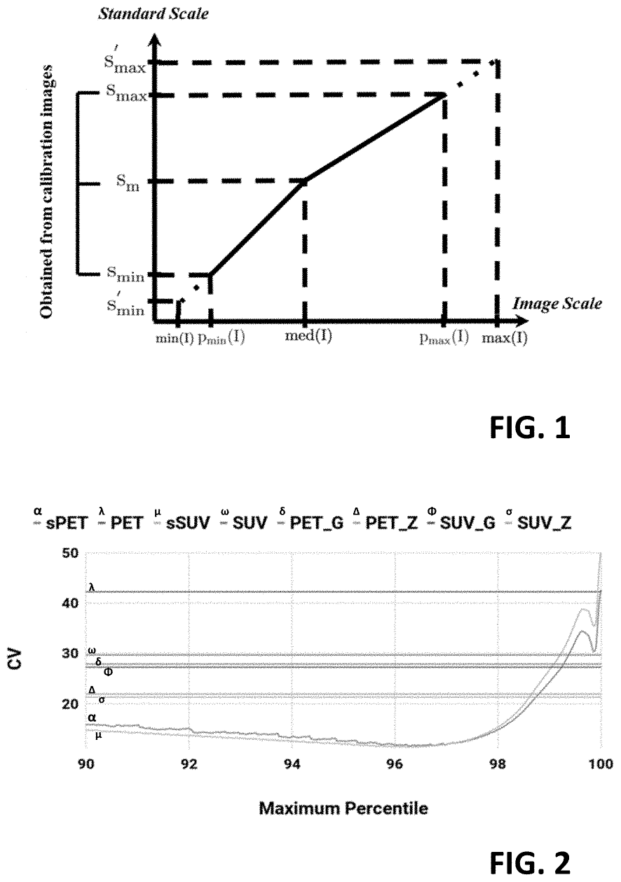

For a given test image I∈I_{T}, in the interval [sₘᵢₙ, sₘₐₓ] intensities get mapped in a non-linear manner as follows. The intensities of I in [pₘᵢₙ(I), med(I)] are mapped linearly to [sₘᵢₙ, sₘ], and similarly intensities of I in [med(I), pₘₐₓ(I)] are mapped to [sₘ,sₘₐₓ]. Then, I's intensities in [min(I), pₘᵢₙ(I)] and [pₘₐₓ(I), max(I)] are mapped, following the mappings from [pₘᵢₙ(I), med(I)] to [sₘᵢₙ′,sₘᵢₙ] and [med(I), pₘₐₓ(I)] to [sₘₐₓ, sₘₐₓ′], respectively (see FIG. 1). FIG. 1 shows mapping a given image to the standard scale. It is important to note that sₘᵢₙ′ and sₘₐₓ′ are calculated based on the obtained landmarks and not from the calibration data set. The formulation of this non-linear mapping is given by the following equation:

$$sI ( v ) = \{ \begin{matrix}\begin{matrix}\frac{{s}_{m} - {s}_{min}}{med ( I ) - {p}_{min} ( I )} ( I ( v ) - \\ {p}_{min} ( I ) ) + {p}_{min} ( I ) ,\end{matrix} & \text{∀} I ( v ) ∈ [ min ( I ) , med ( I ) ] \\ \begin{matrix}\frac{{s}_{max} - {s}_{m}}{{p}_{max} ( I ) - med ( I )} ( I ( v ) - \\ med ( I ) ) + {s}_{m} ,\end{matrix} & \text{∀} I ( v ) ∈ ( med ( I ) , max ( I ) ]\end{matrix} \qquad ( 6 )$$

2.4 Evaluation Methods

CV_{MMA} and CV_{MA} are not only used to find the best pₘₐₓ, but also to serve as metrics for evaluation among images in I_{T}. CV_{MMA} is defined by the following equation:

$${CV}_{MMA} = \frac{{σ}_{MMA}}{{μ}_{MMA}} \qquad ( 7 )$$

where σ_{MMA} and μ_{MMA} are standard deviation and mean, respectively, among MMA's of SUV (sSUV) images in I_{T}. CV_{MA} is similarly defined for PET (sPET) images by the following equation:

$${CV}_{MA} = \frac{{σ}_{MA}}{{μ}_{MA}} \qquad ( 8 )$$

where σ_{MA} and μ_{MA} are standard deviation and mean, respectively, among MA's of PET (sPET) images in I_{T}. In addition to using CV_{MMA} and CV_{MA} as metrics for evaluation, we propose that in repeated scans, the MMA difference for the same organ between initial and repeat scans should decrease after standardization. The Sum of Absolute of Normalized Difference (SD) for 0 of MMA or MA over all images in I_{R} (repeated scan data set) is defined as:

$${SD}_{MMA} ( O ) = {\sum }_{I ∈ {I}_{c}} \frac{\vert {MMA}_{1} ( O ) - {MMA}_{2} ( O ) \vert}{[ {MMA}_{1} ( O ) + {MMA}_{2} ( O ) ] / 2} , \qquad ( 9 )$$

where subscripts 1 and 2 indicate MMA values obtained at the two instances of repeat scans from the same subject.

Normalization is performed in Equation 9 since the values of MMA are in different ranges before and after standardization. SD_{MA} is defined as following:

$${SD}_{MA} ( O ) = {\sum }_{I ∈ {I}_{c}} \frac{\vert {MA}_{1} ( O ) - {MA}_{2} ( O ) \vert}{[ {MA}_{1} ( O ) + {MA}_{2} ( O ) ] / 2} , \qquad ( 10 )$$

where subscripts 1 and 2 indicate MA values obtained at the two instances of repeat scans from the same subject.

Normalization is performed in Equation 10 since the values of MA are in different ranges before and after standardization.

### 3. Data Set and Experiments

3.1 Data Sets

This retrospective study was conducted following approval from the Institutional Review Board at the Hospital of the University of Pennsylvania along with a Health Insurance Portability and Accountability Act waiver. The following data sets were utilized for this study.

Healthy Scan Data Set

This data set includes 38 whole-body FDG-PET/CT scans with normal-appearing livers and spleens (as verified by a board-certified radiologist (co-author Torigian)) including PET/CT scans in 17 women (mean age 69, range 52-85 years) previously acquired on a Biograph mCT scanner (Siemens Healthcare, Erlangen, Germany) and PET/CT scans in 21 men (mean age 44, range 30-50 years) previously acquired on a Gemini TF scanner (Philips Center, Amsterdam, The Netherlands). These 38 scans were acquired approximately 60 minutes after administration of approximately 15 mCi of FDG. This dataset is considered as the normal dataset in the calibration and test phase. About 60% of the whole data set is considered as calibration (I_{S}) and the rest (about 40%) is considered for test (I_{T}).

Repeated Scan Data Set

This data set (IR) includes 23 repeated sets of whole-body FDG-PET/CT scans from 12 men and 11 women (mean age 59, range 40-71 years) with advanced stage non-small cell lung carcinoma previously acquired on Gemini TF (Philips Center, Amsterdam, The Netherlands), Discovery (General Electric Healthcare, Waukesha, Wis.), and Biograph 40 (Siemens Healthcare, Erlangen, Germany) PET/CT scanners. All patients had previously undergone initial and repeat FDG-PET/CT imaging within 7 days without intervening therapy where repeat scans were performed using similar FDG administration and image acquisition parameters as to the initial scans. Both initial and repeat FDG-PET/CT scans had been acquired with FDG uptake delay times within 10-15 minutes of each other. This data set is denoted as IR in our description and it constitutes another test image set.

3.2 Experiments and Results

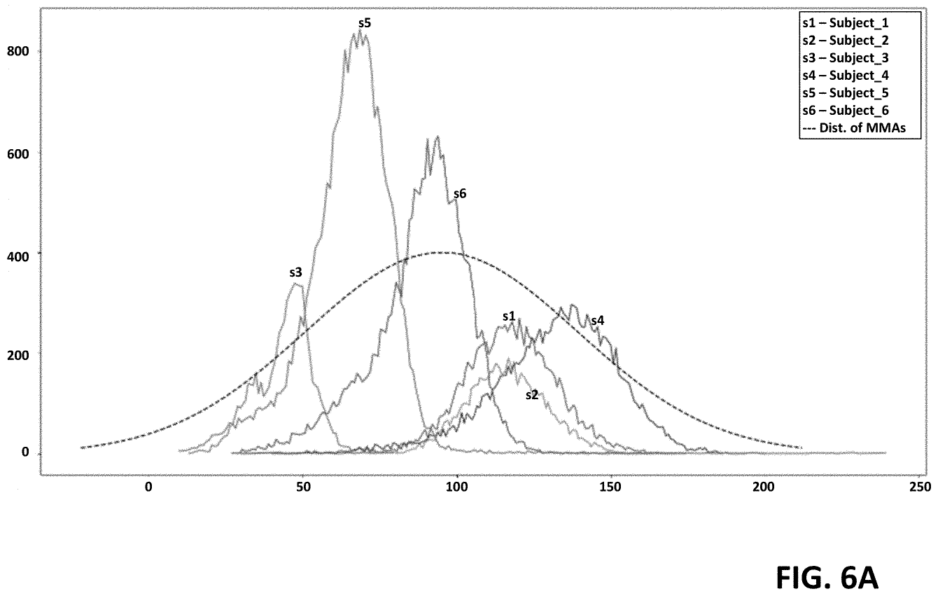

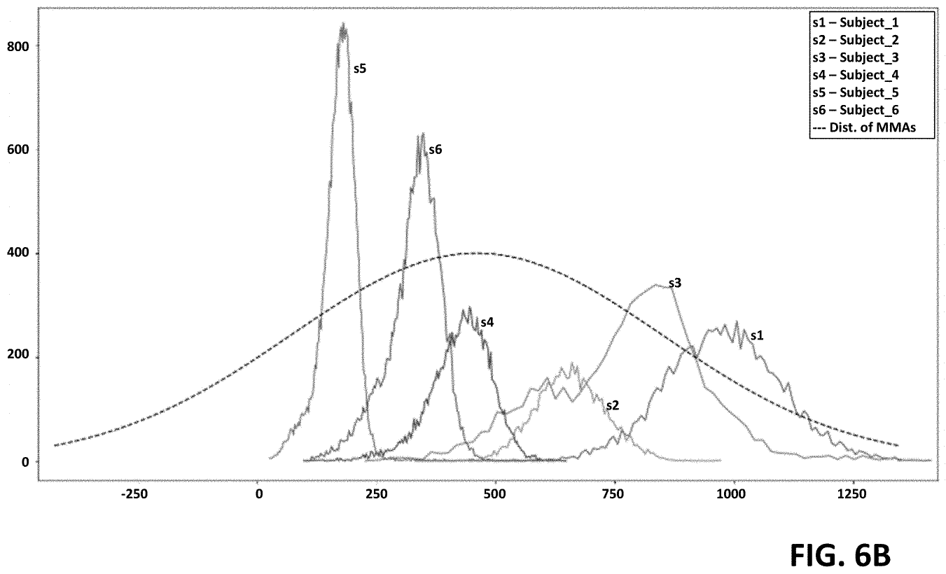

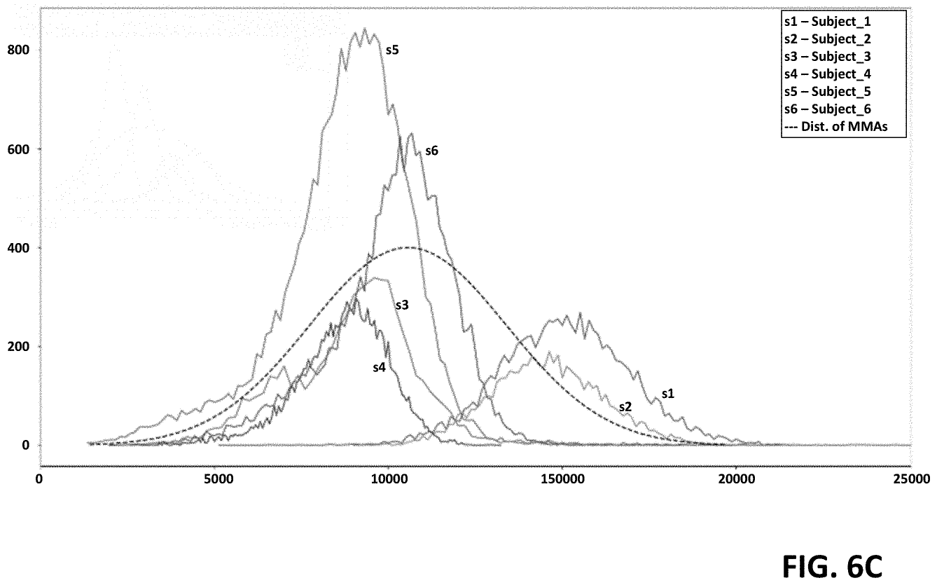

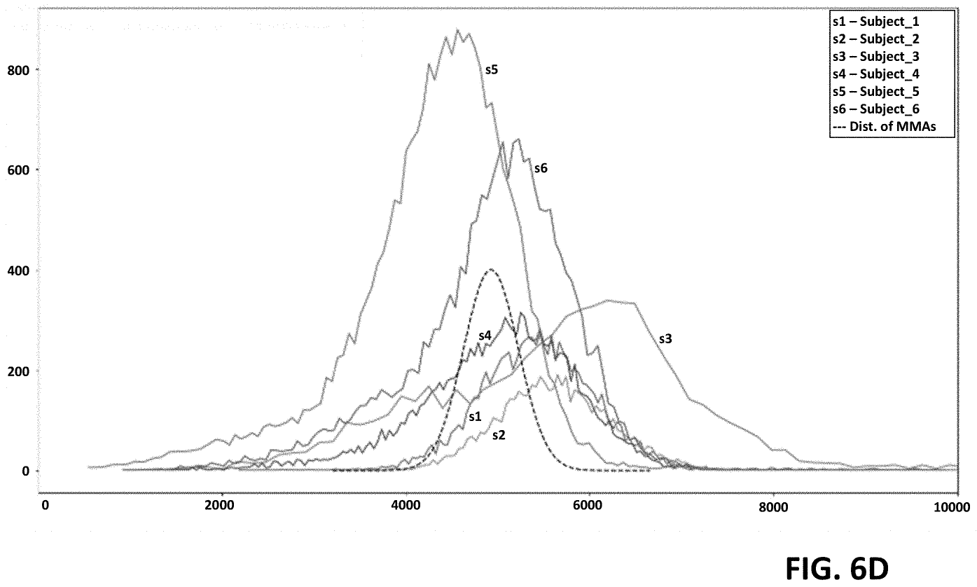

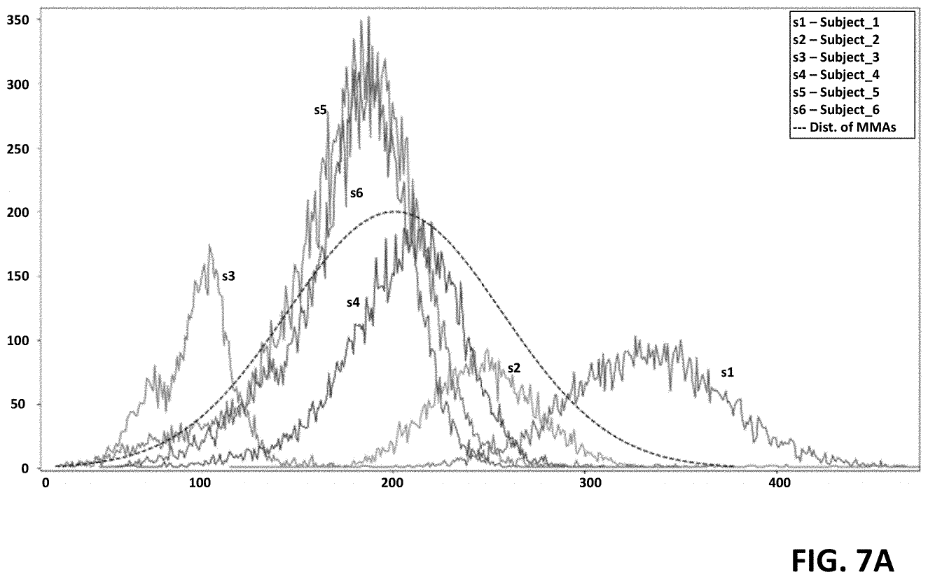

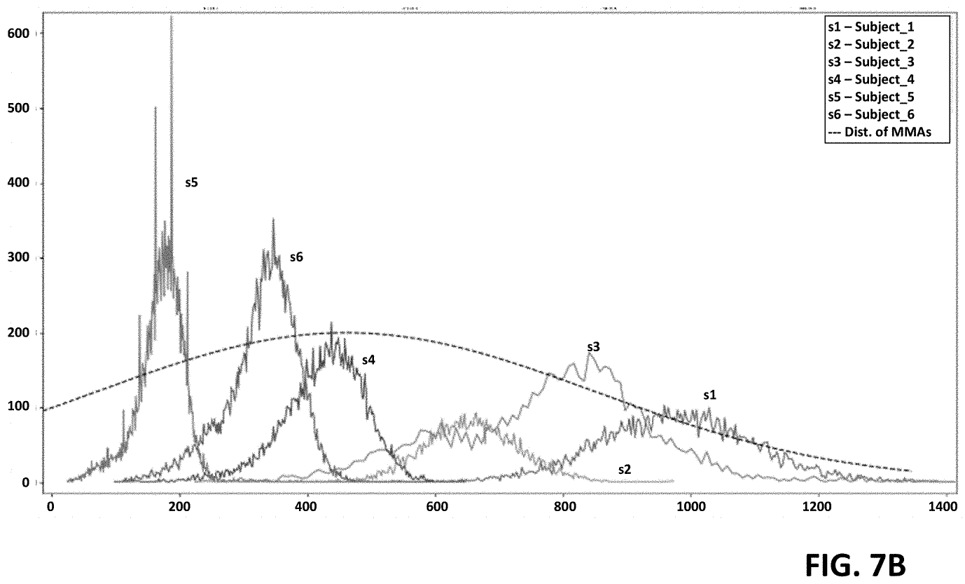

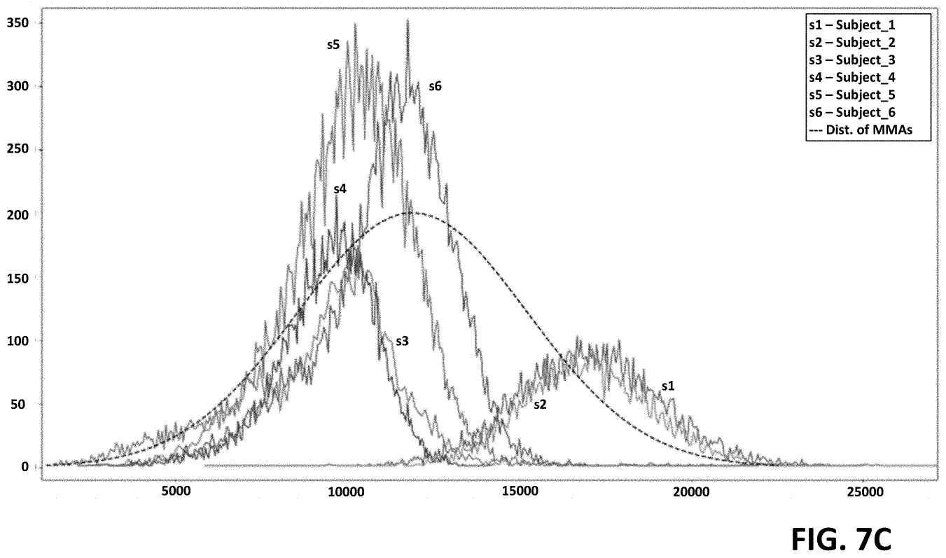

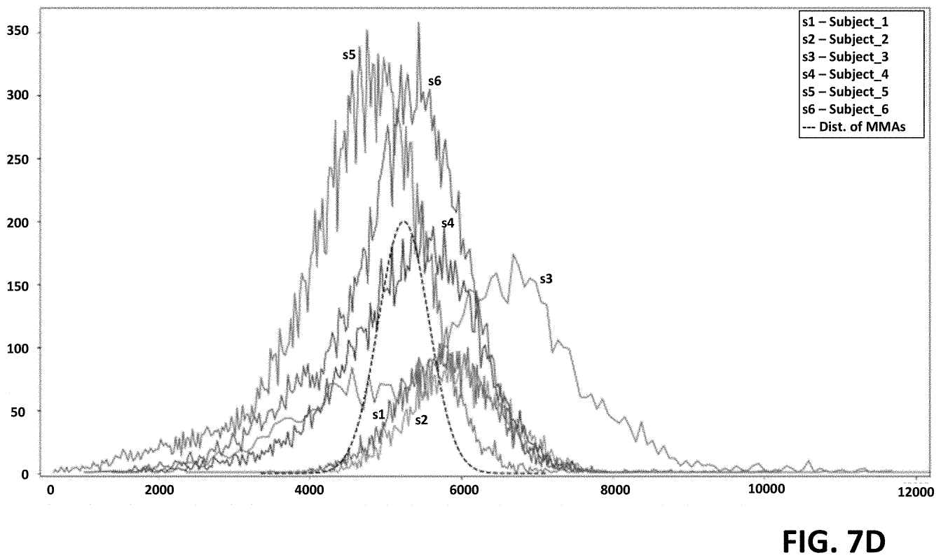

In our experiments, we employ CV_{MMA} and CV_{MA} determined from data set I_{T} to assess the effectiveness of the standardization process. Our results by considering CV_{MMA} and CV_{MA} in data set I_{T} are summarized in Tables 2 and 3 and also their changes versus maximum percentile are illustrated in FIG. 2 and FIG. 3. Also, in order to show how much improvement the proposed method provides, the histogram of livers from some of the subjects from I_{T} are illustrated in FIG. 6 and FIG. 7. FIG. 6A shows the histogram of healthy livers from original PET images. FIG. 6D shows the histogram of livers from sPET. In addition, for fair comparison, we linearly mapped maximum intensity and also 96.5 percentile (optimum value found for pₘₐₓ for PET) of intensities values of each PET image to a fixed value of 6000 and 5000, respectively. The histogram of liver for these linear mappings are illustrated in FIG. 6B and FIG. 6C. In addition, FIG. 7A shows the histogram of healthy livers from original SUV images. FIG. 7D shows the histogram of livers from sSUV. For fair comparison, we linearly mapped maximum intensity and also 95.6 percentile (optimum value found for pₘₐₓ for SUV) of intensities values of each SUV image to a fixed value of 6000 and 5000, respectively. The histogram of liver for these linear mappings are illustrated in FIG. 7B and FIG. 7C. As it can be observed in FIG. 6 and FIG. 7, the means of histograms of livers after using proposed standardization are closer together. Also, another key point in these figures is comparison among the histograms of PET and SUV images. SUV standardization has handled some of the undesired variations in the PET images, but not all of them. As illustrated in FIG. 6A and FIG. 7A, the means of purple (subject_5) and brown (subject_6) histograms come closer together after applying SUV, although the green (subject_3) histogram has not moved that much. This reveals that there are some artifacts in the PET image of green histogram which cannot be removed by the SUV method. However, after applying our proposed standardization method to either PET or SUV image, the green histogram has moved to the mean of all histograms as illustrated in FIG. 6D and FIG. 7D. This shows that sPET method can remove artifacts which cannot be handled by the SUV method.

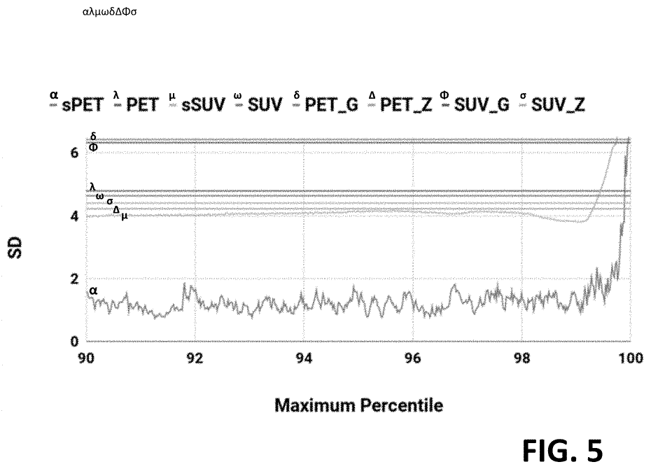

Also, for the repeated scan data set, SD_{MMA} and SD_{MA} versus changes in maximum percentile for liver and spleen are shown in FIGS. 4 and 5, respectively. Here, we assessed MMA and MA for the entire organ, including any lesions if present. Since the underlying whole organ metabolism is not expected to change between repeat scans for the same subject, we expect the MMA and MA values to remain the same between the two scans. Our results for repeated scan data set I_{R} before and after standardization considering the two scans separately and assessed via SD_{MMA} and SD_{MA} are summarized in Table 2 and Table 3.

TABLE 2
CV values for liver and spleen in data set I_{T} before and after standardization.
For PET, optimal pₘₐₓ = 96.4%. For SUV, optimal pₘₐₓ = 95.6%.

| | PET | PET_{G} | PET_{Z} | SUV | SUV_{G} | SUV_{Z} | sPET | sSUV |
|---|---|---|---|---|---|---|---|---|
| | CV_{MA} | CV_{MA} | CV_{MA} | CV_{MMA} | CV_{MMA} | CV_{MMA} | CV_{MA} | CV_{MMA} |
| Liver | 42.28% | 27.29% | 21.62% | 30.10% | 27.28% | 21.51% | 11.56% | 11.78% |
| Spleen | 37.50% | 20.23% | 17.73% | 27.56% | 20.26% | 17.83% | 12.24% | 12.40% |

TABLE 3
SD values for liver and spleen in data set I_{R} before and after
standardization. The value of pₘₐₓ found are as follows. For PET, optimal
pₘₐₓ = 96.4%. For SUV, optimal pₘₐₓ = 95.6%.

| | PET | PET_{G} | PET_{Z} | SUV | SUV_{G} | SUV_{Z} | sPET | sSUV |
|---|---|---|---|---|---|---|---|---|
| | SD_{MA} | SD_{MA} | SD_{MA} | SD_{MMA} | SD_{MMA} | SD_{MMA} | SD_{MA} | SD_{MMA} |
| Liver | 2.15 | 2.38 | 3.38 | 1.88 | 2.40 | 3.66 | 0.59 | 1.02 |
| Spleen | 4.79 | 6.42 | 4.22 | 4.63 | 6.32 | 4.40 | 0.78 | 3.81 |

Also, in order to do a comparison with other similar methods, we apply the Gaussian and Z-score normalization (standardization) methods, as described in [32], to both PET and SUV images. Note that [32] relates to MRI images instead of PET or SUV image standardization. The Gaussian normalized PET image (PET_{G}) regarding organ O can be defined as:

$${PET}_{G} = \frac{PET}{{σ}_{PET}^{O}} \qquad ( 11 )$$

where σ_{PET}^{o} is the standard deviation of organ O in the PET image. The Gaussian normalized SUV image (SUV_{G}) regarding organ O can be defined as:

$${SUV}_{G} = \frac{SUV}{{σ}_{SUV}^{O}} \qquad ( 12 )$$

where σ_{SUV}^{o} is the standard deviation of organ O in the SUV image. The Z-score normalized PET image (PET_{z}) regarding organ O can be defined as:

$${PET}_{z} = \frac{PET - {μ}_{PET}^{O}}{{σ}_{PET}^{O}} \qquad ( 13 )$$

where σ_{PET}^{o} is the mean of the organ O in the PET images. The Z-score normalized SUV image (SUV_{z}) regarding organ O can be defined as:

$${SUV}_{z} = \frac{SUV - {μ}_{SUV}^{O}}{{σ}_{SUV}^{O}} \qquad ( 14 )$$

where μ_{SUV}^{o} is mean of the organ O in the SUV image. The results of these methods regarding each object and each image are shown in Table 2 and Table 3. Our proposed method outperformed other methods in both healthy scan data sets and repeated scan data sets as demonstrated in Table 2 and Table 3. Also, in FIGS. 2-5, the results are compared to each other.

### 4. Discussion and Conclusion

We propose a new method for standardizing PET and SUV images in order to mitigate the effect of undesired factors that impede accurate quantitative analysis of PET images. The strength of the proposed method is shown through different experiments and with different metrics among selected healthy organs and in organs in repeated scans. The proposed method is an adaptation to PET and SUV imagery and further advancement of an established method that introduced image standardization into MR imaging. The results indicate that substantial improvement in the uniformity of numerical meaning is achieved for both PET and SUV images after standardization, and more so for the former. Note that although the subjects included here in I_{c} and I_{T} are considered as healthy based on review of the scans, it is likely that there are differences in the exact health state of individual subjects. This is perhaps one reason, among others, for the residual non-standardness that is left over after standardization has been applied.

The proposed method is superior to other methods in that it is not dependent upon subject and image acquisition related parameters. The sPET method was directly applied to PET images without use of image acquisition-related or patient-related parameters and outperformed other popular methods described in the literature including SUV, Z-score normalization, and Gaussian normalization, based on two new performance metrics called CV and SD. CV was used in healthy scan data sets to measure how similar the metabolic activities of healthy organs were, whereas the SD was used to measure how similar the metabolic activities of organ were between initial and repeated scans. We have shown that our proposed method achieved better results in comparison with other popular methods for removing non-standardness in PET images.

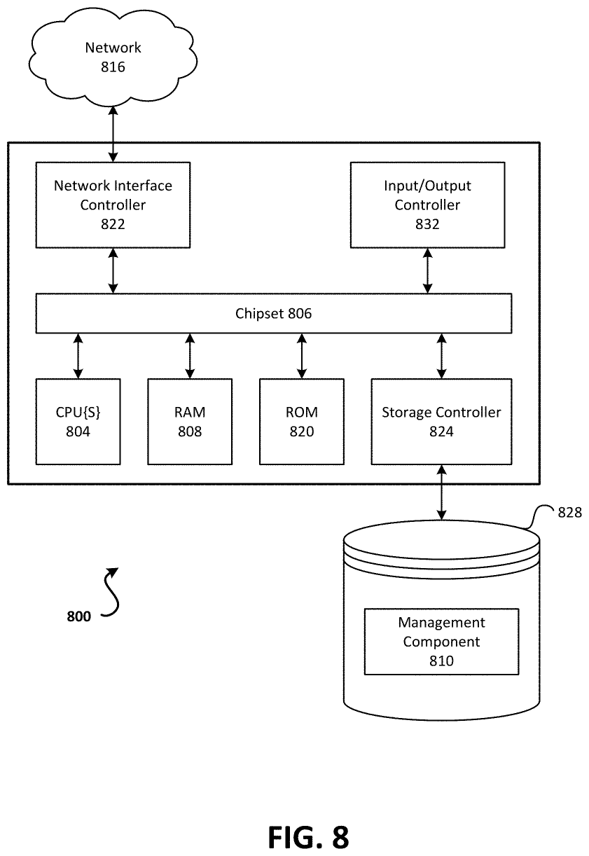

FIG. 8 depicts a computing device that can be used in various aspects, such as the servers, modules, and/or devices depicted elsewhere herein. The computer architecture shown in FIG. 8 shows a conventional server computer, workstation, desktop computer, laptop, tablet, network appliance, PDA, e-reader, digital cellular phone, or other computing node, and can be utilized to execute any aspects of the computers described herein, such as to implement the methods described herein.

The computing device 800 can include a baseboard, or “motherboard,” which is a printed circuit board to which a multitude of components or devices can be connected by way of a system bus or other electrical communication paths. One or more central processing units (CPUs) 804 can operate in conjunction with a chipset 806. The CPU(s) 804 can be standard programmable processors that perform arithmetic and logical operations necessary for the operation of the computing device 800.

The CPU(s) 804 can perform the necessary operations by transitioning from one discrete physical state to the next through the manipulation of switching elements that differentiate between and change these states. Switching elements can generally include electronic circuits that maintain one of two binary states, such as flip-flops, and electronic circuits that provide an output state based on the logical combination of the states of one or more other switching elements, such as logic gates. These basic switching elements can be combined to create more complex logic circuits including registers, adders-subtractors, arithmetic logic units, floating-point units, and the like.

The CPU(s) 804 can be augmented with or replaced by other processing units, such as GPU(s) 805. The GPU(s) 805 can comprise processing units specialized for but not necessarily limited to highly parallel computations, such as graphics and other visualization-related processing.

A chipset 806 can provide an interface between the CPU(s) 804 and the remainder of the components and devices on the baseboard. The chipset 806 can provide an interface to a random access memory (RAM) 808 used as the main memory in the computing device 800. The chipset 806 can further provide an interface to a computer-readable storage medium, such as a read-only memory (ROM) 820 or non-volatile RAM (NVRAM) (not shown), for storing basic routines that can help to start up the computing device 800 and to transfer information between the various components and devices. ROM 820 or NVRAM can also store other software components necessary for the operation of the computing device 800 in accordance with the aspects described herein.

The computing device 800 can operate in a networked environment using logical connections to remote computing nodes and computer systems through local area network (LAN) 816. The chipset 806 can include functionality for providing network connectivity through a network interface controller (NIC) 822, such as a gigabit Ethernet adapter. A NIC 822 can be capable of connecting the computing device 800 to other computing nodes over a network 816. It should be appreciated that multiple NICs 822 can be present in the computing device 800, connecting the computing device to other types of networks and remote computer systems.

The computing device 800 can be connected to a mass storage device 828 that provides non-volatile storage for the computer. The mass storage device 828 can store system programs, application programs, other program modules, and data, which have been described in greater detail herein. The mass storage device 828 can be connected to the computing device 800 through a storage controller 824 connected to the chipset 806. The mass storage device 828 can consist of one or more physical storage units. A storage controller 824 can interface with the physical storage units through a serial attached SCSI (SAS) interface, a serial advanced technology attachment (SATA) interface, a fiber channel (FC) interface, or other type of interface for physically connecting and transferring data between computers and physical storage units.

The computing device 800 can store data on a mass storage device 828 by transforming the physical state of the physical storage units to reflect the information being stored. The specific transformation of a physical state can depend on various factors and on different implementations of this description. Examples of such factors can include, but are not limited to, the technology used to implement the physical storage units and whether the mass storage device 828 is characterized as primary or secondary storage and the like.

For example, the computing device 800 can store information to the mass storage device 828 by issuing instructions through a storage controller 824 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computing device 800 can further read information from the mass storage device 828 by detecting the physical states or characteristics of one or more particular locations within the physical storage units.

In addition to the mass storage device 828 described above, the computing device 500 can have access to other computer-readable storage media to store and retrieve information, such as program modules, data structures, or other data. It should be appreciated by those skilled in the art that computer-readable storage media can be any available media that provides for the storage of non-transitory data and that can be accessed by the computing device 800.

By way of example and not limitation, computer-readable storage media can include volatile and non-volatile, transitory computer-readable storage media and non-transitory computer-readable storage media, and removable and non-removable media implemented in any method or technology. Computer-readable storage media includes, but is not limited to, RAM, ROM, erasable programmable ROM (“EPROM”), electrically erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage, other magnetic storage devices, or any other medium that can be used to store the desired information in a non-transitory fashion.

A mass storage device, such as the mass storage device 828 depicted in FIG. 8, can store an operating system utilized to control the operation of the computing device 800. The operating system can comprise a version of the LINUX operating system. The operating system can comprise a version of the WINDOWS SERVER operating system from the MICROSOFT Corporation. According to further aspects, the operating system can comprise a version of the UNIX operating system. Various mobile phone operating systems, such as IOS and ANDROID, can also be utilized. It should be appreciated that other operating systems can also be utilized. The mass storage device 828 can store other system or application programs and data utilized by the computing device 800.

The mass storage device 828 or other computer-readable storage media can also be encoded with computer-executable instructions, which, when loaded into the computing device 800, transforms the computing device from a general-purpose computing system into a special-purpose computer capable of implementing the aspects described herein. These computer-executable instructions transform the computing device 800 by specifying how the CPU(s) 804 transition between states, as described above. The computing device 800 can have access to computer-readable storage media storing computer-executable instructions, which, when executed by the computing device 500, can perform the methods described herein.

A computing device, such as the computing device 800 depicted in FIG. 8, can also include an input/output controller 832 for receiving and processing input from a number of input devices, such as a keyboard, a mouse, a touchpad, a touch screen, an electronic stylus, or other type of input device. Similarly, an input/output controller 832 can provide output to a display, such as a computer monitor, a flat-panel display, a digital projector, a printer, a plotter, or other type of output device. It will be appreciated that the computing device 800 cannot include all of the components shown in FIG. 8, can include other components that are not explicitly shown in FIG. 8, or can utilize an architecture completely different than that shown in FIG. 8.

As described herein, a computing device can be a physical computing device, such as the computing device 800 of FIG. 8. A computing node can also include a virtual machine host process and one or more virtual machine instances. Computer-executable instructions can be executed by the physical hardware of a computing device indirectly through interpretation and/or execution of instructions stored and executed in the context of a virtual machine.

### Additional Examples

There are two additional developments that make use of the positron emission tomography/standardized uptake value (PET/SUV) standardization techniques: (1) A method to model SUV variation in any given anatomic region such as organs, tissue regions, and lymph node zones. (2) A method of making use of SUV standardization and SUV modeling to perform disease quantification in anatomic regions. These two developments are briefly described below. Without SUV standardization, SUV modeling does not become meaningful. Without SUV standardization and SUV modeling, automatic quantification of disease body-wide is not feasible. Therefore, since SUV standardization techniques with high accuracy are yet to be developed, fully automated body-wide disease quantification techniques are not available at present.

As used herein the general term anatomic region can refer to an organ, tissue region, lymph node zone, an entire body region, or any space within the body that can be defined anatomically consistently and unambiguously.

1) Modeling Normal SUV Variation

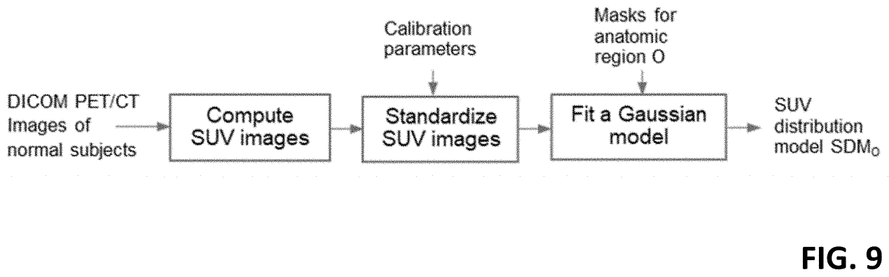

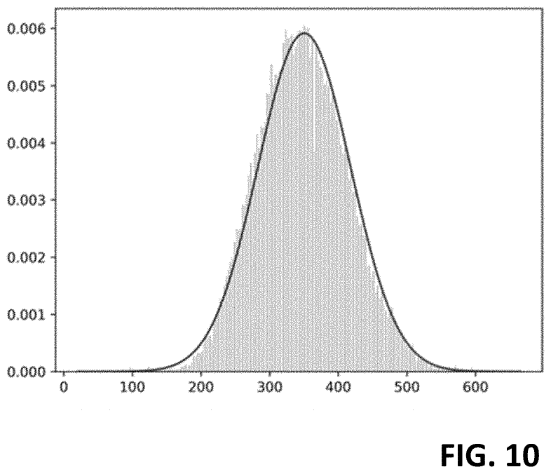

FIG. 9 shows an example SUV modeling process. A set of ¹⁸F-fluorodeoxyglucose (FDG) PET/CT images of normal subjects is gathered. The PET images in the set are then converted to SUV images by using Equation 1. A subset of the SUV images is used for calibration purposes for computing the parameters of the standardization process (e.g., see Section 2.2). Then the SUV images corresponding to the PET images in the input PET/CT acquisitions are standardized following the methods of Section 2.3. Subsequently, a cumulative histogram of the standardized SUVs within the anatomic region O in the SUV images is computed. To this histogram (e.g., probability distribution), a Gaussian function is fit by estimating the mean and standard deviation of the Gaussian from the cumulative histogram. The fit Gaussian function, which we will denote by SDM_{O}(s), is output as an SUV distribution model for O. Note that SDM_{O}(s) indicates the probability density value corresponding to a standardized SUV value s, and this model can be used in estimating disease quantity in O in any given patient PET/CT scan. FIG. 10 shows a cumulative histogram of the standardized SUVs within the esophagus obtained from PET/CT scans of 33 normal subjects and the fit Gaussian function.

(2) Disease Quantification

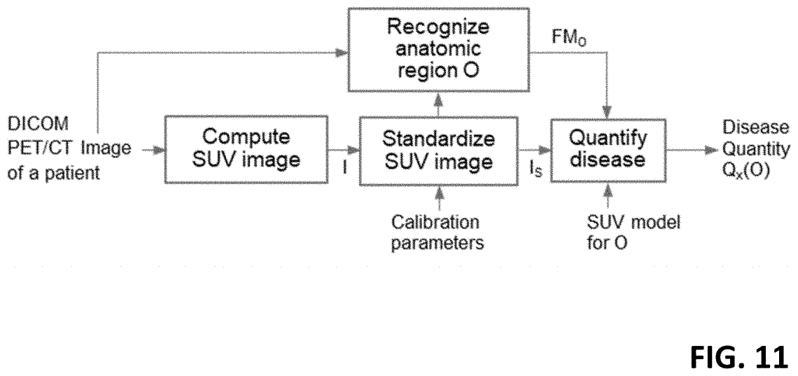

FIG. 11 shows an example disease quantification process. Given a patient PET/CT scan, SUV image I may be computed from the PET image. Image I can be subsequently standardized using the SUV standardization method described in Section 2.3 (e.g., or elsewhere herein). This results in a standardized SUV image I_{S}. Each anatomic region O within which disease quantity is to be estimated can be recognized by using an appropriate object recognition method such as the method of automatic anatomy recognition (AAR) [33, 34]. Object recognition can be performed by utilizing the CT image portion of the PET/CT scan [33], both CT and PET images [33, 35], CT and the standardized SUV image I_{S} [35], PET images alone, or a combination thereof. In all cases, the output of the recognition step can be a mask or an image FM_{O} (e.g., fuzzy model of region O) whose value FM_{O}(v) at any voxel v is 1 if v belongs to O or 0 if v does not belong to O. Alternatively, if a fuzzy recognition method is employed, the voxel can have any fractional value between 0 and 1 indicating the degree of membership of v in O. The next step of disease quantification will make use of FM_{O} in either of these forms, image I_{S}, and the SUV model created for O (e.g., as described above in FIG. 11) to output quantified disease, which can be denoted as Q_{X}(O) in FIG. 11. Q_{X}(O) can comprise (e.g., or consists of) at least three entities for each anatomic region O: Q_{X}(O)=[SUVₘₑₐₙ(O), SUVₘₐₓ(O), TLB(O)]. SUVₘₑₐₙ(O) represents the mean of standardized SUV within O, SUVₘₐₓ(O) denotes the maximum of standardized SUV within O, and TLB(O) represents total lesion burden within O. The computation of SUVmean (O) and SUVₘₐₓ(O) is straightforward once FM_{O} and I_{S} are known. TLB(O) can be estimated as follows:

$$\begin{matrix}TLB ( O ) = TMA ( O ) - {TMA}_{n} ( O ) , & ( 15 ) \\ TMA ( O ) = {\sum }_{v} \vert v \vert \times {FM}_{O} ( v ) \times {I}_{S} ( v ) , & ( 16 ) \\ {TMA}_{n} ( O ) = {\sum }_{v} \vert v \vert \times {FM}_{O} ( v ) \times {NM}_{o} ( {I}_{S} ( v ) ) \times {I}_{S} ( v ) , & \\ {NM}_{O} ( {I}_{S} ( v ) ) = \{ \begin{matrix}{SDM}_{O} ( {I}_{S} ( v ) ) , & if {I}_{S} ( v ) > {μ}_{n} , \\ 1 , & if {I}_{S} ( v ) \geq {μ}_{n} .\end{matrix} & ( 17 )\end{matrix}$$

In Equation (15), TMA(O) denotes the total metabolic activity within O in I_{S}, where |v| denotes volume of voxel v. TMA(O) is simply the sum of the product of the standardized SUV at v within O and the volume of v. The fundamental concept underlying our disease quantification idea is to estimate TMA(O) for O in the patient image I_{S} by using Equation (16), and subtract from it the contribution of normal tissue activity, denoted by TMAₙ(O), that would ensue if O were normal, to obtain Total Lesion Burden TLB(O) of O in I_{S}. The idea is to retain only the disease portions of the radiotracer activity and to remove the background normal radiotracer activity, without explicitly segmenting either the lesions or O, by employing fuzzy set principles. The process also enables us to estimate (in a fuzzy manner) the normal and pathologic portions of the tissues within O. Note the difference in the meaning of TMA(O) and TLB(O). This subtle difference is the essence of the underlying main idea for disease quantification. Note also that we expect TLB(O) for a healthy/normal organ to hover around O, the deviation from O denoting the various approximations and errors that enter into the entire disease quantification process starting from the first stage of image acquisition and potential normal variation.

The idea underlying the estimation of TMAₙ(O) in Equation (17) is to consider the SUV distribution SDM_{O}(s) defined for the normal population of O. For an SUV value (random variable value) s=I_{S}(v) at a voxel v in the SUV image I_{S} of a normal organ O, a normality map NM_{O}(s) may be determined based on SDM_{O}(s) such that NM_{O}(s) expresses the likelihood of s denoting normal tissue of O at voxel v. The likelihood may be employed as a weight value (e.g., fuzzy membership value) to modulate the contribution of v to TMAₙ(O) as expressed in Equation (17). The parameter μₙ in Equation (17) denotes the mean of the Gaussian distribution function SDM_{O}(s). Note that without SUV standardization, the SUV distribution may not be modeled reliably as is evident from FIGS. 6 and 7, tissue normality of O may not be ascertained reliably, and therefore disease burden may not be estimated reliably. It should be noted that in place of the Gaussian functional form for SDM_{O}, we may use any other functions that are appropriate for the SUV distribution of normal O. Other more sophisticated functions can also be fit to the cumulative histogram using techniques such as Kernel Density Estimation and employed in Equation 17. In this case, μₛ will represent the mode of the function.

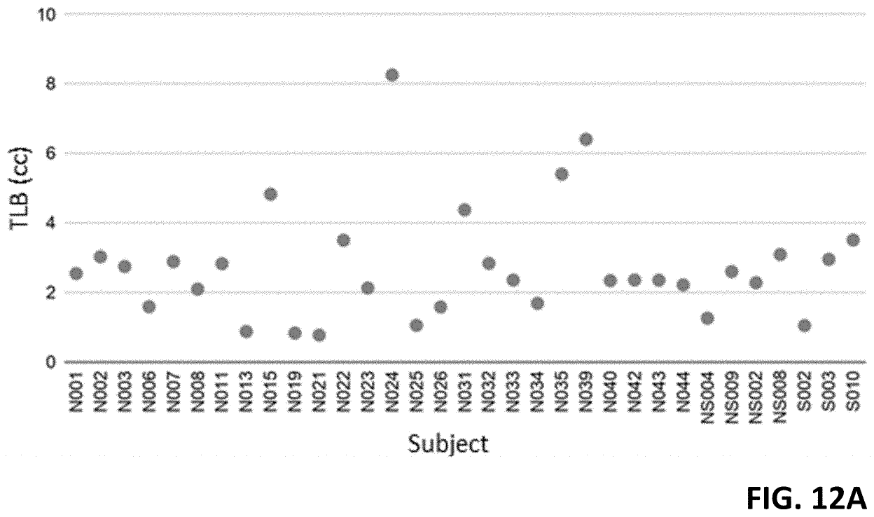

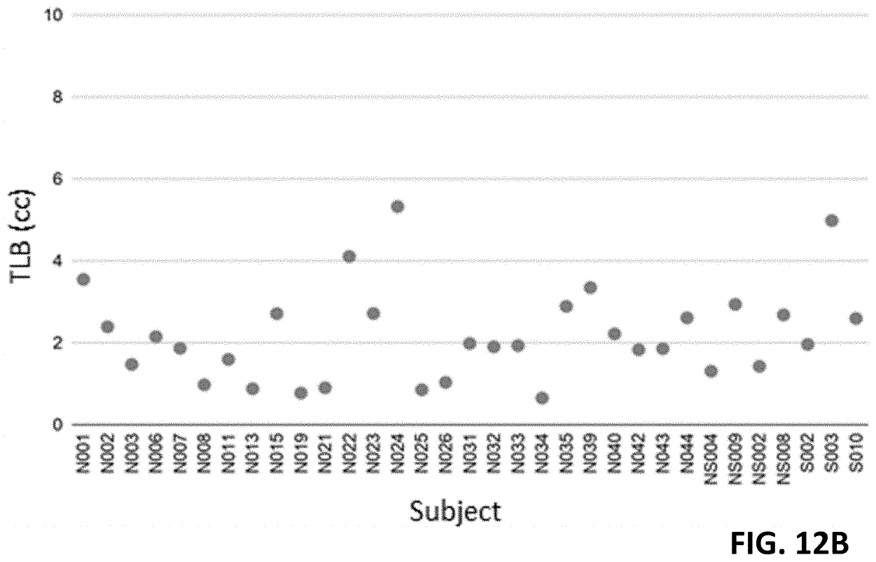

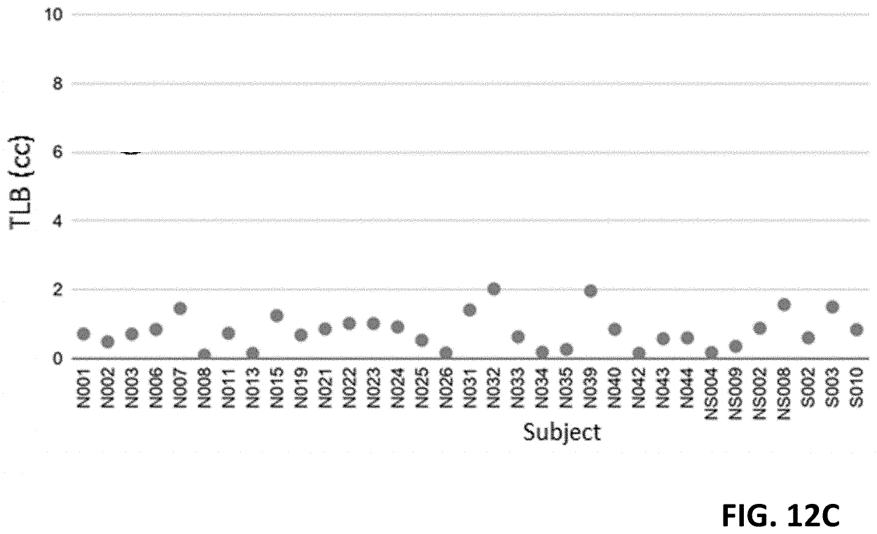

To illustrate and validate how the disease quantification process works, we show in FIGS. 12A-C TLB(O) estimated from PET/CT scans of 33 normal subjects for three organs, namely right lung, left lung, and esophagus. FIG. 12A shows total lesion burden for normal subjects for the right lung. FIG. 12B shows total lesion burden for normal subjects for the left lung. FIG. 12C shows total lesion burden for normal subjects for the esophagus. The different subject scans are indicated along the horizontal axis and the estimated TLB(O) is depicted along the vertical axis. Clearly, the method correctly estimates the disease quantity (total lesion burden) to be near 0, where the deviation from 0 denotes the variations in normality that can be expected in the normal population.

Please note that this entire methodology can also be similarly applied to PET images that have been acquired using radiotracers other than FDG, as well as to PET images acquired from PET/magnetic resonance imaging (MRI) scanners and from standalone PET scanners.

It is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Components are described that can be used to perform the described methods and systems. When combinations, subsets, interactions, groups, etc., of these components are described, it is understood that while specific references to each of the various individual and collective combinations and permutations of these may not be explicitly described, each is specifically contemplated and described herein, for all methods and systems. This applies to all aspects of this application including, but not limited to, operations in described methods. Thus, if there are a variety of additional operations that can be performed it is understood that each of these additional operations can be performed with any specific embodiment or combination of embodiments of the described methods.

As will be appreciated by one skilled in the art, the methods and systems can take the form of an entirely hardware embodiment, an entirely software embodiment, or an embodiment combining software and hardware aspects. Furthermore, the methods and systems can take the form of a computer program product on a computer-readable storage medium having computer-readable program instructions (e.g., computer software) embodied in the storage medium. More particularly, the present methods and systems can take the form of web-implemented computer software. Any suitable computer-readable storage medium can be utilized including hard disks, CD-ROMs, optical storage devices, or magnetic storage devices.

Embodiments of the methods and systems are described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions can be loaded on a general-purpose computer, special-purpose computer, or other programmable data processing apparatus to produce a machine, such that the instructions which execute on the computer or other programmable data processing apparatus create a means for implementing the functions specified in the flowchart block or blocks.

These computer program instructions can also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

The various features and processes described above can be used independently of one another, or can be combined in various ways. All possible combinations and sub-combinations are intended to fall within the scope of this disclosure. In addition, certain methods or process blocks can be omitted in some implementations. The methods and processes described herein are also not limited to any particular sequence, and the blocks or states relating thereto can be performed in other sequences that are appropriate. For example, described blocks or states can be performed in an order other than that specifically described, or multiple blocks or states can be combined in a single block or state. The example blocks or states can be performed in serial, in parallel, or in some other manner. Blocks or states can be added to or removed from the described example embodiments. The example systems and components described herein can be configured differently than described. For example, elements can be added to, removed from, or rearranged compared to the described example embodiments.

It will also be appreciated that various items are illustrated as being stored in memory or on storage while being used, and that these items or portions thereof can be transferred between memory and other storage devices for purposes of memory management and data integrity. Alternatively, in other embodiments, some or all of the software modules and/or systems can execute in memory on another device and communicate with the illustrated computing systems via inter-computer communication. Furthermore, in some embodiments, some or all of the systems and/or modules can be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures can also be stored (e.g., as software instructions or structured data) on a computer-readable medium, such as a hard disk, a memory, a network, or a portable media article to be read by an appropriate device or via an appropriate connection. The systems, modules, and data structures can also be transmitted as generated data signals (e.g., as part of a carrier wave or other analog or digital propagated signal) on a variety of computer-readable transmission media, including wireless-based and wired/cable-based media, and can take a variety of forms (e.g., as part of a single or multiplexed analog signal, or as multiple discrete digital packets or frames). Such computer program products can also take other forms in other embodiments. Accordingly, the present invention can be practiced with other computer system configurations.

While the methods and systems have been described in connection with preferred embodiments and specific examples, it is not intended that the scope be limited to the particular embodiments set forth, as the embodiments herein are intended in all respects to be illustrative rather than restrictive.

It will be apparent to those skilled in the art that various modifications and variations can be made without departing from the scope or spirit of the present disclosure. Other embodiments will be apparent to those skilled in the art from consideration of the specification and practices described herein. It is intended that the specification and example figures be considered as exemplary only, with a true scope and spirit being indicated by the following claims.

The disclosure can comprise at least the following aspects.

Aspect 1. A method, comprising, consisting of, or consisting essentially of: receiving a plurality of images based on positron emission tomography; determining, based on the plurality of images, a plurality of calibration parameters indicative of standardized intensity values for corresponding percentiles of intensity values; determining at least one image associated with a patient; applying, based on the plurality of calibration parameters, a transformation to the at least one image associated with the patient; and providing the transformed at least one image.

Aspect 2. The method of Aspect 1, wherein determining the plurality of calibration parameters comprises determining a standardized maximum percentile intensity value.

Aspect 3. The method of Aspect 2, wherein the standardized maximum percentile intensity value is determined based on intensity values above the standardized maximum percentile intensity value varying greater than a threshold amount among the plurality of images.

Aspect 4. The method of any one of Aspects 2-3, wherein the standardized maximum percentile intensity value comprises a value in a range of one or more of about 85 to about 100, about 90 to about 100, about 95 to about 100, about 95 to about 95, or about 96 to about 97.

Aspect 5. The method of any one of Aspects 2-3, wherein applying the transformation comprises transforming intensity values of the at least one image based one or more ranges defined based on the plurality of calibration parameters.

Aspect 6. The method of Aspect 5, wherein intensity values of the at least one image that are above the standardized maximum percentile intensity value are transformed based on a range defined by a standardized median percentile value of the plurality of calibration parameters and the standardized maximum percentile intensity value.

Aspect 7. The method of any one of Aspects 2-6, wherein the plurality of calibration parameters comprises a standardized minimum percentile intensity value and a standardized median percentile intensity value.

Aspect 8. The method of Aspect 7, wherein applying the transformation comprises: determining, based on the at least one image associated with the patient, a patient specific minimum percentile intensity value, a patient specific median percentile intensity value, and a patient specific maximum percentile intensity value.

Aspect 9. The method of Aspect 8, wherein applying the transformation comprises: determining a first range of intensity values of the at least one image based on the patient specific minimum percentile intensity value and the patient specific median percentile intensity value; determining a second range of intensity values of the at least one image based on the patient specific median percentile intensity value and the patient specific maximum percentile intensity value; mapping intensity values of the at least one image in the first range to a first calibrated range defined by the standardized minimum percentile intensity value and the standardized median percentile intensity value; mapping intensity values of the at least one image in the second range to a second calibrated range defined by the standardized median percentile intensity value and the standardized maximum percentile intensity value; and mapping intensity values of the at least one image above the patient specific maximum percentile intensity value to the second calibrated range.

Aspect 10. The method of any one of Aspects 1-9, wherein determining the plurality of calibration parameters comprises: determining, for each of the plurality of images, statistical intensity values indicating different percentiles of intensity values; and determining the plurality of calibration parameters by averaging, for each of the different percentiles, the determined statistical intensity values.

Aspect 11. The method of any one of Aspects 1-10, wherein the plurality of images comprise images of a plurality of different patients.

Aspect 12. The method of any one of Aspects 1-11, wherein the plurality of images comprises positron emission tomography (PET) images.

Aspect 13. The method of any one of Aspects 1-12, wherein the plurality of images comprises standardized uptake value (SUV) images.

Aspect 14. The method of any one of Aspects 1-13, wherein the plurality of calibration parameters are specific to one or more of a type of radiation used for the positron emission tomography or a type of radiotracer used for the positron emission tomography.

Aspect 15. The method of any one of Aspects 1-14, wherein the plurality of calibration parameters are specific to an imaging device used to generate the plurality of images.

Aspect 16. The method of any one of Aspects 1-15, wherein the plurality of images are generated using a same imaging device, and wherein the at least one image is generated using the imaging device.

Aspect 17. The method of any one of Aspects 1-16, wherein providing the transformed at least one image comprising one or more of storing the transformed at least one image, causing output of the transformed at least one image, or transmitting the at least one image.

Aspect 18. The method of any one of Aspects 1-17, further comprising determining, based on a model associated with an anatomic region, an indication of a disease burden associated with the transformed image.

Aspect 19. The method of Aspect 18, wherein the model is based on a plurality of images transformed based on the one or more calibration parameters.

Aspect 20. The method of Aspect 18, wherein the model and the disease burden is specific to an anatomic region indicated based on a mask.

Aspect 21. A method, comprising, consisting of, or consisting essentially of: determining an image of a subject, wherein the image has intensity values that are standardized based on one or more calibration parameters; determining an indication of an anatomic region in the image; determining, based on the indication of the anatomic region and a model associated with the anatomic region, an indication of a disease burden associated with the image; and causing output of the indication of the disease burden associated with the image.

Aspect 22. The method of Aspect 21, further comprising: determining a plurality of images associated with a plurality of subjects and calibrated based on the one or more calibration parameters; and determining, based on the plurality of images, the model associated with the anatomic region.

Aspect 23. The method of Aspect 22, further comprising receiving a mask indicating the anatomic region, and wherein determining the model of the anatomic region comprises applying the mask to the plurality of images.

Aspect 24. The method of Aspect 22, wherein determining the model comprises determining one or more parameters of the model based on fitting at least a portion of a plurality of images to the model.

Aspect 25. The method of any one of Aspects 21-24, wherein the model is a gaussian model.

Aspect 26. The method of any one of Aspects 21-25, wherein the model indicates a standard distribution of values for subjects with normal tissue.

Aspect 27. The method of any one of Aspects 21-26, wherein the indication of the anatomic region comprises one or more of a mask or a fuzzy mask to apply to the image.

Aspect 28. The method of any one of Aspects 21-27, where determining the anatomic region comprises one or more of determining the anatomic region based on one or more of a machine learning model, a fuzzy object model, or data indicative of user input.

Aspect 29. The method of any one of Aspects 21-28, wherein determining the indication of the disease burden comprises subtracting a normal activity metric representing normal tissue for the anatomic region from a total activity metric representing tissue from the image for the anatomic region.

Aspect 30. The method of Aspect 29, wherein the normal activity metric is based on summing intensity values based on the model, and wherein the total activity metric is based on summing intensity values for voxels of the image.

Aspect 31. The method of any one of Aspects 29-30, further comprising determining the total activity metric by summing, for a plurality of voxels of the image, a product of: a volume of the corresponding voxel, a value of a mask corresponding to the voxel, and an intensity value of the image for the corresponding voxel.

Aspect 32. The method of any one of Aspects 29-31, further comprising: determining, based on the model, a normality map indicating likelihood of normal tissue at a plurality of voxels; and determining the normal activity metric by summing, for at least a portion of the plurality of voxels, a product of: a volume of the corresponding voxel, a value of a mask corresponding to the voxel, a value of the normality map corresponding to the voxel, and an intensity value for the corresponding voxel.

Aspect 33. The method of any one of Aspects 21-32, wherein the image comprises one or more of a positron emission tomography or a standardized uptake value image.

Aspect 34. The method of any one of Aspects 21-33, wherein causing output of the indication of the disease burden associated with the image comprises one or more of sending the indication to a device, causing storage of the indication of the disease burden, or causing output via a display of the indication of the disease burden.

Aspect 35. The method of any one of Aspects 21-34, wherein determining the image comprises one or more of: receiving the image, or generating the image based on the one or more calibration parameters and an initial image.

Aspect 36. The method of any one of Aspects 21-35, wherein the one or more calibration parameters comprise one or more of the plurality of calibration parameters of any one of Aspects 1-20.

Aspect 37. A method comprising, consisting of, or consisting essential of: determining a plurality of images associated with a plurality of subjects and calibrated based on one or more calibration parameters; determining, based on the plurality of images, a model associated with the anatomic region; and causing output of one or more of the model or data based on the model.

Aspect 38. The method of Aspect 37, further comprising receiving a mask indicating the anatomic region, and wherein determining the model of the anatomic region comprises applying the mask to the plurality of images.

Aspect 39. The method of any one of Aspects 37-38, wherein determining the model comprises determining one or more parameters of the model based on fitting at least a portion of a plurality of images to the model.

Aspect 40. The method of any one of Aspects 37-39, wherein the one or more calibration parameters comprise one or more of the plurality of calibration parameters of any one of Aspects 1-20.

Aspect 41. The method of any one of Aspects 37-40, wherein the data based on the model comprise an indication of a disease burden.

Aspect 42. The method of any one of Aspects 37-41, wherein causing output of the model comprises causing output of one or more of a visual representation of the model, a graph of the model, or a parameter of the model.

Aspect 43. A device comprising, consisting of, or consisting essentially of: one or more processors; and a memory storing instructions that, when executed by the one or more processors, cause the device to perform the methods of any one of Aspects 1-42.

Aspect 44. A non-transitory computer-readable medium storing instructions that, when executed by one or more processors, cause a device to perform the methods of any one of Aspects 1-42.

Aspect 45. A system, comprising, consisting of, or consisting essentially of: an imaging device configured to perform positron emission tomography; a memory storing instructions that, when executed by the one or more processors, cause the system to perform the methods of any one of Aspects 1-42.

### BIBLIOGRAPHY

[1] L. Nyul and J. Udupa, “On standardizing the MR image intensity scale,” Magnetic Resonance in Medicine: An Official Journal of the International Society for Magnetic Resonance in Medicine, pp. 1072-1081, 1999.
[2] L. Nyul, J. Udupa and X. Zhang, “New variants of a method of MRI scale standardization,” IEEE transactions on medical imaging, pp. 143-150, 2000.
[3] R. Siegel, K. Miller and J. A., “Cancer statistics, 2019,” CA: a cancer journal for clinicians, pp. 7-34, 2019.
[4] T. Kwee, S. Basu, B. Saboury, A. Alavi and D. Torigian, “Functional oncoimaging techniques with potential clinical applications,” Frontiers in bioscience (Elite edition), pp. 1081-1096, 2012.
[5] T. Kwee, D. Torigian and A. Alavi, “Overview of positron emission tomography, hybrid positron emission tomography instrumentation, and positron emission tomography quantification,” Journal of thoracic imaging 28, pp. 4-10, 2013.
[6] T. Kwee, D. Torigian and A. Alavi, “Oncological applications of positron emission tomography for evaluation of the thorax,” Journal of thoracic imaging 28, pp. 11-24, 2013.
[7] T. Kwee, D. Torigian and A. Alavi, “Nononcological applications of positron emission tomography for evaluation of the thorax.,” Journal of thoracic imaging, 28, pp. 25-39, 2013.
[8] B. Bai, J. Bading and P. Conti, “Tumor quantification in clinical positron emission tomography,” Theranostics 3, no. 10, 2013.
[9] P. S. Conti and L. Strauss, “The applications of PET in clinical oncology,” J Nucl Med 32, pp. 623-648, 1991.
[10] S. Basu, H. Zaidi, M. Houseni, G. Bural, J. Udupa, P. Acton, D. Torigian and A. Alavi, “Novel quantitative techniques for assessing regional and global function and structure based on modern imaging modalities: implications for normal variation, aging and diseased states,” Seminars in nuclear medicine, vol. 37, no. 3, pp. 223-239, 2007.
[11] J. W. Keyes Jr, “SUV: standard uptake or silly useless value?,” Journal of Nuclear Medicine 36, pp. 1836-1839, 1995.
[12] C. Brendle, J. Kupferschlager, K. Nikolaou, C. Fougère, S. Gatidis and C. Pfannenberg, “I_{S} the standard uptake value (SUV) appropriate for quantification in clinical PET imaging?—Variability induced by different SUV measurements and varying reconstruction methods,” European journal of radiology 84, pp. 158-162, 2015.
[13] M. Vanderhoek, S. Perlman and J. R., “Impact of the definition of peak standardized uptake value on quantification of treatment response,” ournal of Nuclear Medicine 53, pp. 4-11, 2012.
[14] B. Thomas, K. Erlandsson, M. Modat, L. Thurfjell, L. Vandenberghe, S. Ourselin and H. B. F., “The importance of appropriate partial volume correction for PET quantification in Alzheimers disease,” European journal of nuclear medicine and molecular imaging 38, pp. 1104-1119, 2011.
[15] Y. Wang, E. Chiu, J. Rosenberg and G. S. S., “Standardized uptake value atlas: characterization of physiological 2-deoxy-2-[18 F] fluoro-d-glucose uptake in normal tissues,” Molecular Imaging and Biology 9, pp. 83-90, 2007.
[16] B. Kurland, L. Peterson, A. Shields, J. Lee, D. Byrd, A. Novakova-Jiresova, M. Muzi, J. Specht, D. Mankoff, H. Linden and P. Kinahan, “Test-Retest Reproducibility of 18F-FDG PET/CT Uptake in Cancer Patients Within a Qualified and Calibrated Local Network,” Journal of Nuclear Medicine 60.5, pp. 608-614, 2019.
[17] E. Pfaehler, R. Beukinga, J. de Jong, R. Slart, C. Slump, R. Dierckx and R. Boellaard, “Repeatability of 18F-FDG PET radiomic features: A phantom study to explore sensitivity to image reconstruction settings, noise, and delineation method,” Medical physics, 46(2), pp. 665-678.
[18] A. Ferretti, S. Chondrogiannis, L. Rampin, E. Bellan, M. Marzola, G. Grassetto, S. Gusella, A. Maffione, M. Gava and D. Rubello, “How to harmonize SUVs obtained by hybrid PET/CT scanners with and without point spread function correction,” Physics in Medicine & Biology, 63(23), p. 235010, 2018.
[19] D. Rubello and P. Colletti, “SUV harmonization between different hybrid PET/CT systems,” Clinical nuclear medicine, 43(11), pp. 811-814, 2018.
[20] C. Sprinz, S. Altmayer, M. Zanon, G. Watte, K. Irion, E. Marchiori and B. Hochhegger, “Effects of blood glucose level on 18F-FDG uptake for PET/CT in normal organs: a systematic review,” PloS one, 13(2), p. 0193140, 2018.
[21] B. Altazi, G. Zhang, D. Fernandez, M. Montejo, D. Hunt, J. Werner, M. Biagioli and E. Moros, “Reproducibility of F18-FDG PET radiomic features for different cervical tumor segmentation methods, gray-level discretization, and reconstruction algorithms,” Journal of applied clinical medical physics, 18(6), pp. 32-48, 2017.
[22] H. Engel, H. Steinert, A. Buck, T. Berthold, R. Boni and S. G. K., “Whole-body PET: physiological and artifactual fluorodeoxyglucose accumulations,” Journal of Nuclear Medicine 37, pp. 441-445, 1996.
[23] H. Bae, J. Tsuchiya, T. Okamoto, I. Ito, Y. Sonehara, F. Nagahama, K. Kubota and U. Tateishi, “Standardization of [F-18] FDG PET/CT for response evaluation by the Radiologic Society of North America-Quantitative Imaging Biomarker Alliance (RSNAQIBA) profile: preliminary results from the Japan-QIBA (J-QIBA) activities for Asian international multice,” Japanese journal of radiology 36, p. 686-690, 2018.
[24] M. Namias, T. Bradshaw, V. Menezes, M. Machado and J. R, “A novel approach for quantitative harmonization in PET,” Physics in Medicine & Biology 63, p. 095019, 2018.
[25] F. DiFilippo, M. Patel and S. Patel, “Automated Quantitative Analysis of American College of Radiology PET Phantom Images,” Journal of Nuclear Medicine Technology, pp. 149-254, 2019.
[26] T. Hara, T. Kobayashi, S. Ito, X. Zhou, T. Katafuchi and H. Fujita, “Quantitative analysis of torso FDG-PET scans by using anatomical standardization of normal cases from thorough physical examinations,” PloS one 10, p. e0125713, 2015.
[27] M. Scarpelli, J. Eickhoff, E. Cuna, S. Perlman and R. Jeraj, “Optimal transformations leading to normal distributions of positron emission tomography standardized uptake values,” Physics in Medicine & Biology 63, p. 035021, 2018.
[28] F. Orlhac, S. Boughdad, C. Philippe, H. Stalla-Bourdillon, C. Nioche, L. Champion, M. Soussan, F. Frouin, V. Frouin and I. Buvat, “A postreconstruction harmonization method for multicenter radiomic studies in PET,” Journal of Nuclear Medicine, 59(8), pp. 1321-1328, 2018.
[29] T. Nakahara, H. Daisaki, Y. Yamamoto, T. Iimori, K. Miyagawa, T. Okamoto, Y. Owaki, N. Yada, K. Sawada, R. Tokorodani and M. Jinzaki, “Use of a digital phantom developed by QIBA for harmonizing SUVs obtained from the state-of-the-art SPECT/CT systems: a multicenter study,” EJNMMI research, 7(1), p. 53, 2017.
[30] A. Madabhushi and U. J. K., “New methods of MR image intensity standardization via generalized scale,” Medical physics 33, pp. 3426-3434, 2006.
[31] Y. Zhuge, J. Udupa, J. Liu and P. Saha, “Image background inhomogeneity correction in MRI via intensity standardization,” Computerized Medical Imaging and Graphics 33, pp. 7-16, 2009.
[32] B. Ellingson, T. Zaw, T. Cloughesy, K. Naeini, S. Lalezari, S. Mong, A. Lai, P. Nghiemphu and W. Pope, “Comparison between intensity normalization techniques for dynamic susceptibility contrast (DSC)-MRI estimates of cerebral blood volume (CBV) in human gliomas,” Journal of Magnetic Resonance Imaging, vol. 35.6, pp. 1472-1477, 2012.
[33] Wu X, Udupa J K, Tong Y, Odhner D, Pednekar G V, Simone II C B, McLaughlin D, Apinorasethkul C, Lukens J, Mihailidis D, Shammo G, James P, Camaratta J, Torigian D A. AAR-RT—A system for auto-contouring organs at risk on CT images for radiation therapy planning: Principles, design, and large-scale evaluation on head-and-neck and thoracic cancer cases, Medical Image Analysis, 54: 45-62, 2019. https://doi.org/10.1016/j.media.2019.01.008.
[34] Agrawal V, Udupa J K, Tong Y, Torigian D A. BRR-Net: A CNN-RNN Tandem Architecture for Automatic Body Region Localization in CT Images, Medical Physics, 47 (10): 5020-5031, 2020. https://doi.org/10.1002/MP.14439.
[35] Wang, H., Udupa, J. K., Odhner, D., Tong, Y., Zhao, L., Torigian, D. A.: “Automatic anatomy recognition in whole-body PET/CT images,” Medical Physics, 43(1): 613-629, 2016.

What is claimed:

1. A method, comprising:
receiving a plurality of images based on positron emission tomography;
determining, based on the plurality of images, a plurality of calibration parameters indicative of standardized intensity values for corresponding percentiles of intensity values;
determining at least one image associated with a patient;
applying, based on the plurality of calibration parameters, a transformation to the at least one image associated with the patient; and
providing the transformed at least one image.

2. The method of claim 1, wherein determining the plurality of calibration parameters comprises determining a standardized maximum percentile intensity value.

3. The method of claim 2, wherein the standardized maximum percentile intensity value is determined based on intensity values above the standardized maximum percentile intensity value varying greater than a threshold amount among the plurality of images.

4. The method of claim 2, wherein the standardized maximum percentile intensity value comprises a value in a range of one or more of about 85 to about 100, about 90 to about 100, about 95 to about 100, about 95 to about 95, or about 96 to about 97.

5. The method of claim 2, wherein applying the transformation comprises transforming intensity values of the at least one image based one or more ranges defined based on the plurality of calibration parameters.

6. The method of claim 5, wherein intensity values of the at least one image that are above the standardized maximum percentile intensity value are transformed based on a range defined by a standardized median percentile value of the plurality of calibration parameters and the standardized maximum percentile intensity value.

7. The method of claim 2, wherein the plurality of calibration parameters comprises a standardized minimum percentile intensity value and a standardized median percentile intensity value.

8. A method, comprising:
determining an image of a subject, wherein the image has intensity values that are standardized based on one or more calibration parameters;
determining an indication of an anatomic region in the image;
determining, based on the indication of the anatomic region and a model associated with the anatomic region, an indication of a disease burden associated with the image; and
causing output of the indication of the disease burden associated with the image.

9. The method of claim 8, further comprising:
determining a plurality of images associated with a plurality of subjects and calibrated based on the one or more calibration parameters; and
determining, based on the plurality of images, the model associated with the anatomic region.

10. The method of claim 9, further comprising receiving a mask indicating the anatomic region, and wherein determining the model of the anatomic region comprises applying the mask to the plurality of images.

11. The method of claim 9, wherein determining the model comprises determining one or more parameters of the model based on fitting at least a portion of a plurality of images to the model.

12. The method of claim 8, wherein the model is a gaussian model.

13. The method of claim 8, wherein the model indicates a standard distribution of values for subjects with normal tissue.

14. The method of claim 8, wherein the indication of the anatomic region comprises one or more of a mask or a fuzzy mask to apply to the image.

15. A method comprising:
determining a plurality of images associated with a plurality of subjects and calibrated based on one or more calibration parameters;
determining, based on the plurality of images, a model associated with the anatomic region; and
causing output of one or more of the model or data based on the model.

16. The method of claim 15, further comprising receiving a mask indicating the anatomic region, and wherein determining the model of the anatomic region comprises applying the mask to the plurality of images.

17. The method of claim 15, wherein determining the model comprises determining one or more parameters of the model based on fitting at least a portion of a plurality of images to the model.

18. The method of claim 15, wherein the one or more calibration parameters comprise one or more of the plurality of calibration parameters of any one of claims 1-20.

19. The method of claim 15, wherein the data based on the model comprise an indication of a disease burden.

20. The method of claim 15, wherein causing output of the model comprises causing output of one or more of a visual representation of the model, a graph of the model, or a parameter of the model.
